(19) United States
(12) Patent Application Publication
(10) Pub. No.: US 2024/0315747 A1
(43) Pub. Date: Sep. 26, 2024

(54) BONE STABILIZATION DEVICE

(71) Applicant: ExsoMed Corporation, Aliso Viejo, CA (US)

(72) Inventors: Lloyd P. Champagne, Phoenix, AZ (US); Jozef Zoldos, Phoenix, AZ (US)

(21) Appl. No.: 18/733,142

(22) Filed: Jun. 4, 2024

Related U.S. Application Data
(62) Division of application No. 17/448,184, filed on Sep. 20, 2021.
Application No. 17/448,184 is a continuation of application No. 14/993,972, filed on Jan. 12, 2016, now Pat. No. 11,147,604.

(51) Int. Cl. A61B 17/86; A61B 17/00; A61B 17/72; A61B 17/88; A61F 2/42

(52) U.S. Cl. CPC A61B 17/8645; A61B 17/7291; A61B 17/8615; A61B 17/8635; A61B 17/864; A61F 2/4241; A61B 2017/00867; A61B 17/8897

(57) ABSTRACT

A device and method for stabilizing a broken bone while it heals is disclosed. The device preferably has a (a) first (or proximal) section with a driving head, threads and a first diameter, and (b) second (or distal) section that is threaded and has a second diameter. The first section is preferably greater in diameter than the second section so that greater torque can be applied to tighten the device. The device may include one or more self-tapping structures to lessen the torque required to screw it into a bone.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57.

The present application is a divisional of U.S. patent application Ser. No. 17/448,184, filed Sep. 20, 2021, entitled “BONE STABILIZATION DEVICE,” which is a continuation of U.S. patent application Ser. No. 14/993,972, filed Jan. 12, 2016, entitled “BONE STABILIZATION DEVICE,” the entirety of each of which is hereby incorporated by reference herein.

### FIELD OF THE INVENTION

The present invention relates to a device implantable in a bone to stabilize it while it heals, and which is particularly suitable for use in a metacarpal bone.

### FIELD OF THE INVENTION

The palm of the hand is made up of bones called metacarpals, and a metacarpal connects each finger and thumb to the hand. Each finger and thumb is formed of bones called phalanges. The connection of the phalanges to the metacarpals is called a “knuckle” joint or metacarpophalangeal joint (MCP joint), and acts like a hinge when the fingers or thumb are bent. In the metacarpal bones, the proximal portion and mid metacarpal portion is relatively narrower, and the distal portion is relatively wider with respect to both the internal medullary canal and external diameter.

In each finger, there are three phalanges that are separated by two joints called the interphalangeal joints (IP joints). The proximal IP joint (PIP joint) is the one closest to the MCP joint. The other joint closest to the end of the finger is the distal IP joint (DIP joint). The thumb just has one IP joint. The joints are covered on the ends with articular cartilage.

Damage to the metacarpal bone may occur as a result of a sprain or fracture. Typically, once the metacarpal bone is lined up after an injury it must be stabilized in position while it heals.

To stabilize a broken metacarpal bone, it is now known to use a non-threaded, smooth metal shaft (hereafter “nail”) positioned in the metacarpal bone to hold it in position while the bone heals. An opening is first formed in the metacarpal bone, wherein the opening extends through the fracture and the nail is positioned in the opening to provide lateral stability for the parts of the bone on either side of the fracture. After a certain period, a second surgery is required to remove the nail from the bone. Problems with the nail are that, because it is not anchored in the bone, it can migrate through the metacarpal bone and into surrounding tissue. Sometimes this can result in damage to soft tissue, such as a severed or damaged tendon or cartilage, and/or cause pain. Another problem with the nail is that, because it can migrate, a second surgery is required to remove it. Additionally, the proximal end of pins and nails can cause tendon irritation, tendon rupture or skin irritation and infection.

One potential solution to this problem is to insert a screw into the bone. Such a procedure could be lengthy, and there would be a possibility of bone damage, or damage to the driving head of the screw, which could prevent complete insertion of the screw into the bone, or breakage of the screw because the screw must be relatively long and slender. Current screws are not designed specifically for intramedullary placement. They are not long enough, and if a current screw design was simply lengthened, it would lack a shaft and driving portion sufficient to handle the torque required.

### SUMMARY OF THE INVENTION

The present invention solves the problems associated with repairing a bone, such as a metacarpal bone, by providing a device that is a screw having a first (or proximal in relation to the screw) section and a second (or distal in relation to the screw) section. As used herein with respect to a device, or section of a device, according to the invention, “diameter” includes the diameter of the threads, unless otherwise specified. The first section preferably has a greater diameter than the second section, and the end (or tip) of the first section has a driving surface (or hand) to receive and be turned by an appropriate driving tool. The screw is inserted into the metacarpal in a retrograde fashion. In this manner, the smaller diameter second section can fit in the narrower proximal and mid portion of a bone, such as a metacarpal bone. The larger diameter first section fits into the larger, distal portion of the bone (such as a metacarpal head), and the driving head at the end of the first section is wider in order to accept a larger driving tool and generate more torque to drive the screw into position.

In preferred embodiments, the device has a cutting structure at the tip of the second end, and a second cutting structure between the first section and second section. The cutting structures (either one, two or more, if used) assist in placing the screw into the bone with less torque.

It is also preferred, but not required, that there is an immediate step from the thicker diameter of the first section to the thinner diameter of the second section, and that the second cutting structure be positioned immediately before, or be included as part of, the initial threads in the first section, and that the cutting structure be approximately the same diameter as the first section.

In one exemplary embodiment, the second section is completely threaded (which as used herein means substantially completely threaded) and has an overall outer diameter (including the threads) of 4 mm. The first section is also preferably completely threaded (which as used herein means substantially completely threaded) and has an overall outer diameter of 4.5 mm. The first section, however, may have a diameter of 3.0 mm-5.0 mm, and the second section may have a diameter of 3.0 mm-4.5 mm. It is preferred that the diameter of the first section be about 0.5 mm greater than the diameter of the second section. The first section may instead have a diameter that is 7%-15% greater than the diameter of the second section.

The threads most preferably run along the entire length (which means substantially the entire length) of the device. The threads may have the same pitch and height along the length of the device, or the threads on the first section may have a different pitch and/or height than the threads on the second section.

Because of the configuration of device according to the invention, the device may be inserted and retained in a bone such as the metacarpal. The device generates sufficient fixation to the bone, is thin enough to fit into the proximal and middle portions of the bone, and strong enough so that torque applied to it threads the device into the bone rather than causing the device to deform.

### BRIEF DESCRIPTION OF THE DRAWINGS

FIG. 1 is a top view of the head of one embodiment of a device according to the invention.

FIG. 2 is a cross-sectional side view of the device of FIG. 1.

FIG. 3 is a perspective, side view of the device of FIGS. 1 and 2.

FIG. 4 is a partial, cross-sectional side view of the device of FIGS. 1-3.

FIG. 5 is an end view of the device of FIGS. 1-4.

FIG. 6 is a partial, cross-sectional side view of the device of FIGS. 1-5.

FIGS. 7-7B show one method for installing a device according to FIGS. 1-6 into a metacarpal bone.

FIGS. 7C and 8 illustrate the device of FIGS. 1-6 positioned in a metacarpal bone.

FIG. 9 illustrates a side view of the device of FIGS. 1-6 with a cutting flute shown without threads for clarity.

FIG. 10 illustrates a detailed view of a portion of the device of FIG. 9 with a distal cutting flute shown without threads for clarity.

FIG. 11 illustrates a detailed view of a portion of the device of FIG. 9 with a proximal cutting flute shown without threads for clarity.

### DETAILED DESCRIPTION OF PREFERRED EMBODIMENTS

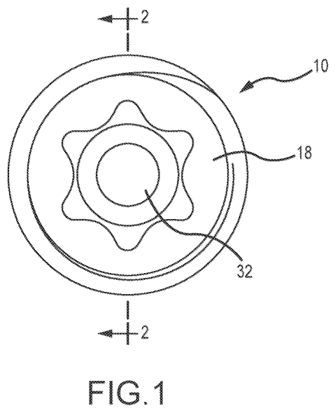

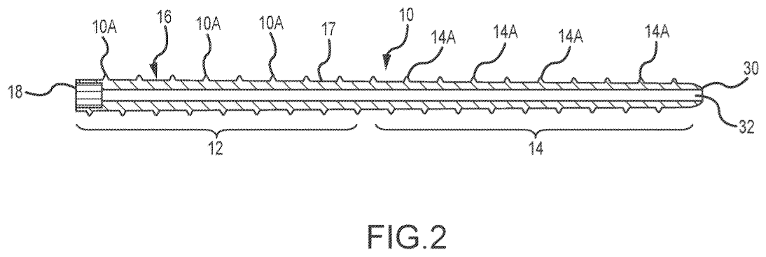

Turning now to the figures, where the purpose is to describe preferred embodiments of the invention and not to limit same, FIG. 1 shows an exemplary embodiment 10 of the invention. Device 10 may be formed of any suitable material, such as titanium steel, stainless steel or nitinol. Device 10 has a first (or proximal) section 12, a second (or distal) section 14, and a shaft 16 with an outer surface 17. Device 10 may be between 3.5 cm and 8.5 cm or between 6.5 cm and 8 cm in length, or have a length of about 7 cm.

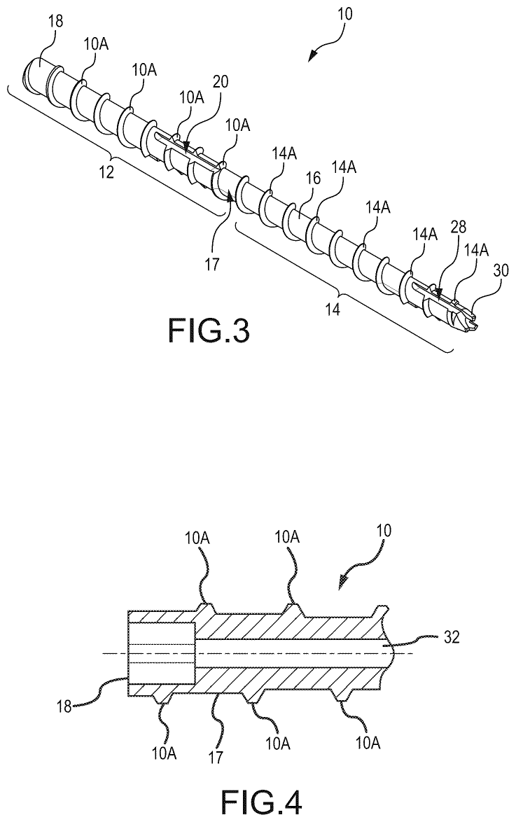

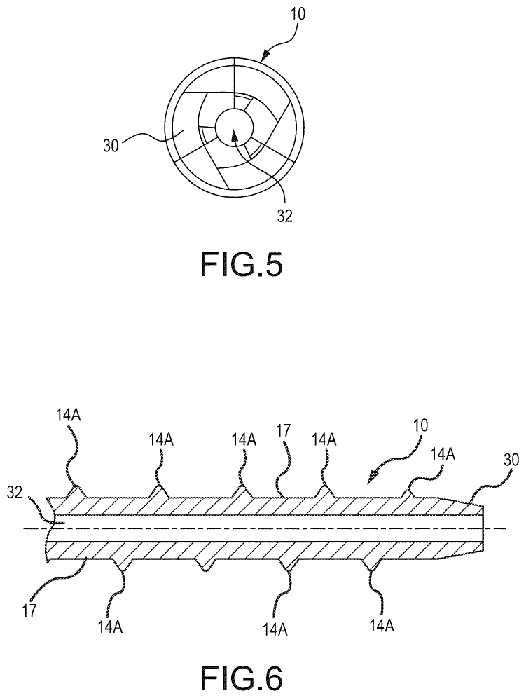

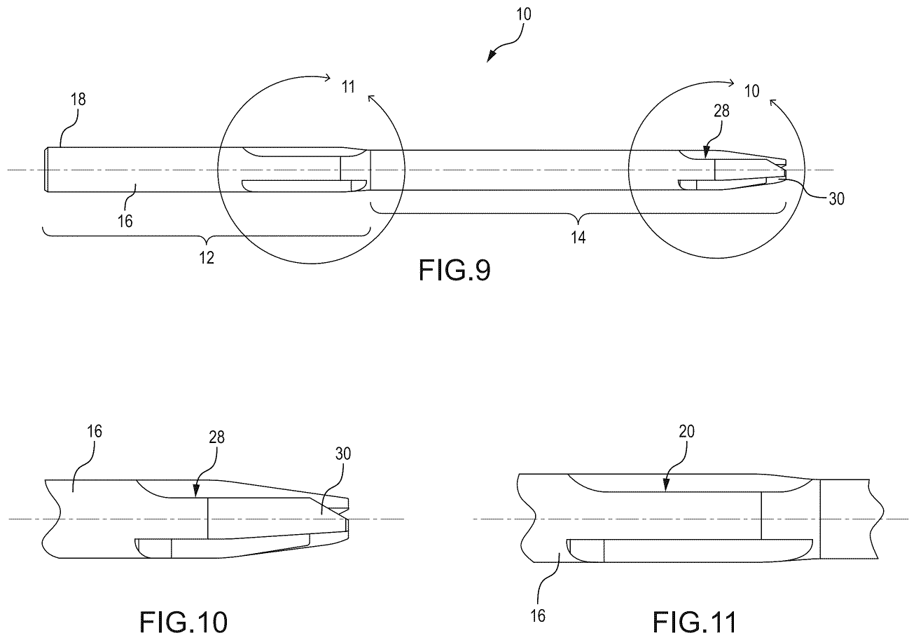

First section 12 has first threads 10A which preferably have a height of about 0.5 to 1 mm as measured from outer surface 17, and a pitch of about 1 mm per revolution. A driving surface, or head 18 is shown as being the same diameter of first section 12, but head 18 may have a different diameter or be of a different shape, such as triangular. Head 18 may accept any suitable driver configuration, such as a Torx drive, slotted, Pozidriv, Robertson, tri-wing, Torq-Set, SpannerHead, Triple Square, and hex head. A cutting structure 20 is shown in FIGS. 3, 9, and 11 as positioned at the distal end of first section 12, and it preferably has the same height as threads 10A, or is formed in one or more threads 10A, and the portion of section 12 that includes cutting structure 20 as shown preferably has the same diameter as the rest of first section 12. The preferred configuration of cutting structure 20 is self-tapping features in one or more threads 10A, which are preferably indentations that basically make the thread serrated, as shown in FIG. 3. First section 12 preferably has a diameter of 3.0 mm to 5.0 mm, and most preferably about 4.5 mm. First section 12 preferably extends between about 25% to 45% of the length of device 10, and in one preferred embodiment is about 40% of the length.

Second section 14 has first threads 14A which preferably have a height of about 0.5-1 mm as measured from outer surface 17, and a pitch of about 1 mm/revolution. A cutting structure 28 is shown in FIGS. 3, 9, and 10 at the distal tip 30 of second section 12, which may be any known self-tapping feature. Second section 14 preferably has a diameter of 3.0 mm to 4.5 mm, most preferably about 4.0 mm, and most preferably about 0.5 mm less than the diameter of the first section. Second section 14 preferably extends between about 55% to 75% of the length of device 10, and in one preferred embodiment is about 60% of the length.

Threads 10A on the first section 10 preferably have the same pitch as the thread 14A, and extend outward from surface 17 of device 10 by the same amount as threads 14A, although any suitable thread configuration is acceptable, including threads with differential pitches threads on either or both of sections 12 and 14.

Device 10 may be cannulated or non-cannulated. As shown, device 10 has a cannula 32 extending therethrough. A non-cannulated device may be have a smaller diameter than a cannulated device.

The diameter of the first section is preferably about 4%, or 5%, or 4% to 7%, or 4%to 10%, of the length of device 10.

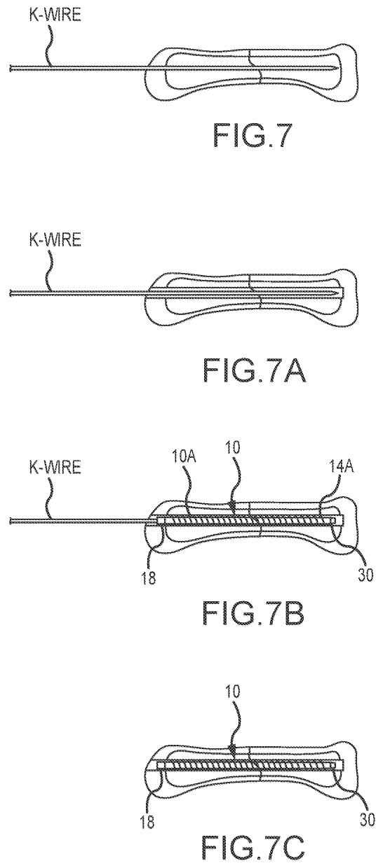

FIGS. 7-7B depict a method for installing device 10 into a fractured metacarpal bone. In FIG. 7, the fracture in the bone is first aligned, and then a K-wire is inserted into the bone. AK-wire or pin is known in the art and is a sterilized, smooth steel pin used in orthopedics and other types of medical applications. It is available in different sizes as needed and provides structure, support and in one version has a diameter of about 0.040″.

In FIG. 7A a cannulated drill, using the K-wire as a guide, drills an opening into the metacarpal bone, wherein the opening extends through the fracture, or partially through the k wired construct, and provides enough space on each side of the fracture to properly position device 10.

In FIG. 7B, device 10 is rotatingly driven into the opening in the metacarpal bone using cannulated techniques. Second section 14 is first driven in, and then first section 12 is driven in. The width of second section 14 and first section 12 are both greater than the opening formed in the bone, so each section can thread into the opening. This provides bone material for the threads to grasp and retain device 10 in the opening.

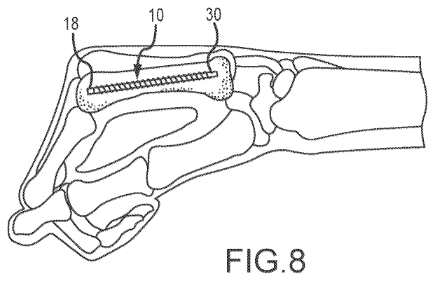

FIGS. 7C and 8 show device 10 in the metacarpal bone after the K-wire has been removed. Since device 10 is anchored in the metacarpal bone, there is no need for a second operation to remove it.

If a non-cannulated device is used, the K wire may be used to form a pilot hole, and device 10 would be driven into the pilot hole.

In another aspect of the invention, the device has a single diameter with threads of the same pitch and height. This device may have a head similar to the one described above, but that is about 0.5 mm wider than the rest of the device, or about 7%-15% wider, in order to generate sufficient torque. Such a device may also have multiple self-tapping, or cutting structures, in order to reduce the amount of torque required to screw the device into an opening in a bone. For example, such a device may have one cutting structure at its distal tip, and one or more other cutting structures along its length, and/or a cutting structure juxtaposed the head so the head of the device does not extend beyond the bone.

Specific exemplary embodiments of the invention are described below:
1. A device for repairing a bone, the device for being received in the bone and comprising:
   (a) a shaft having a length and an outer surface;
   (b) a first end and a second end;
   (c) threads on the outer surface, wherein the threads comprise 85% or more of the shaft length; and
   (d) a driving surface at the first end,
   wherein the first section has a larger diameter than the second section of the device.
2. The device of example 1 wherein the device is comprised of one or more of nitinol, stainless steel and titanium steel.
3. The device of example 1 or 2, wherein the first section has a self-tapping configuration and the second section has a self-tapping portion.
4. The device of any of examples 1-3 wherein the first end has a diameter at least 7% wider than the diameter of the second end.
5. The device of any of examples 1-4 wherein the first end has a diameter that is 7%-15% wider than the diameter of the second end.
6. The device of any of examples 1-5 that has that has a length of between 3.5 cm and 8.5 cm, or 3.5 to 7.5 cm, or 4.0 to 8.0 cm, or about 7 cm.
7. The device of any of examples 1-6 wherein the first diameter is between 3.5 mm and 5.0 mm.
8. The device of any of examples 1-7 wherein the second diameter is between 3.0 mm and 4.5 mm.
9. The device of any of examples 1-8 that further includes a cutting surface at the second end.
10. The device of any of examples 1-9 that further includes a cutting surface between the first end and the second end.
11. The device of any of examples 1-10 that includes a cannula.
12. The device of any of examples 1-11 wherein the first end is between 0.3 mm and 0.7 mm greater in diameter than the diameter of the second end.
13. The device of any of examples 1-12 wherein the first end is between 7% and 15% greater in diameter than the diameter of the second end.
14. The device of any of examples 1-13 wherein the threads on the first end have the same pitch as a pitch of the threads on the second end.
15. The device of any of examples 1-13 wherein the threads on the first end have a different pitch than a pitch of the threads on the second end.
16. The device of any of examples 1-15 wherein the threads are continuous along the length of the device.
17. The device of any of examples 1-16 wherein the threads on the first section comprise up to 40% of the length of the device.
18. The device of any of examples 1-17 wherein the driving head is selected from the group consisting of designs that can accept one of the following drivers: a flat screwdriver, a Phillips screw driver, a hex head, and an Allen wrench.
19. The device of any of examples 1-8 wherein the driving head has the same diameter as the first end.
20. The device of any of examples 1-19 wherein the threads have the same height.
21. The device of any of examples 1-20 wherein the threads have the same height and pitch.
22. The device of any of examples 1-21 that has a length and the diameter of the first section is at least 4%, or at least 5%, of the length.
23. The device of any of examples 1-21 that has a length and the diameter of the first section is between 4% and 7% of the length.
24. The device of any of examples 1-21 that has a length and the diameter of the first section is between 4% and 10% of the length.

Having thus described some embodiments of the invention, other variations and embodiments that do not depart from the spirit of the invention will become apparent to those skilled in the art. The scope of the present invention is thus not limited to any particular embodiment, but is instead set forth in the appended claims and the legal equivalents thereof. Unless expressly stated in the written description or claims, the steps of any method recited in the claims may be performed in any order capable of yielding the desired result.

What is claimed is:

1. A method of implanting a cannulated intramedullary implant within an intramedullary canal of a metacarpal for fixation of a fracture, the method comprising:
inserting a guidewire into the intramedullary canal of the metacarpal;
drilling an opening into the metacarpal past a location of the fracture guided by the guidewire;
rotatably driving the cannulated intramedullary implant into the intramedullary canal guided by the guidewire, the implant comprising a headless shaft having a trailing end and a leading end, the shaft comprising a thread of a constant pitch along an entire length of the shaft, wherein a first portion of the thread has a greater constant major diameter and a second portion of the thread has a smaller constant major diameter, the first portion of the thread being continuous with the second portion of the thread, and wherein the first portion of the thread engages bone material in a larger distal portion of the metacarpal and the second portion of the thread engages bone material in a narrower proximal and mid portion of the metacarpal such that the cannulated intramedullary implant is anchored in the metacarpal; and
removing the guidewire.

2. The method of claim 1, wherein the cannulated intramedullary implant is inserted with the leading end entering the opening into the intramedullary canal first, the second portion of the thread being closer to the leading end than to the trailing end.

3. The method of claim 1, wherein the first portion of the thread engages the metacarpal head.

4. The method of claim 1, wherein a first root diameter of the shaft at the first portion of the thread is greater than a second root diameter of the shaft at the second portion of the thread.

5. The method of claim 4, wherein the first portion and the second portion of the thread are of the same thread height.

6. The method of claim 4, wherein the shaft further comprises a transition region, a root diameter of the transition region transitions from the first root diameter to the second root diameter.

7. The method of claim 1, wherein the shaft comprises a driving head at the trailing end, the driving head configured to receive a driver that applies a torque to rotatably drive the cannulated intramedullary implant into the intramedullary canal.

8. The method of claim 1, wherein the shaft comprises at least one cutting structure extending through part of the first portion of the thread and at least another cutting structure extending through part of the second portion of the thread.

9. The method of claim 1, wherein rotatably driving the cannulated intramedullary implant is performed using a driver, the implant comprising a driving head at the trailing end, wherein the driving head is configured to receive the driver.

10. The method of claim 9, wherein the driver is a flat screwdriver, a Phillips screwdriver, a hex head, or an Allen wrench.

11. A method of implanting a cannulated intramedullary implant within an intramedullary canal of a metacarpal for fixation of a fracture, the method comprising:
inserting a guidewire into the intramedullary canal of the metacarpal;
drilling an opening into the metacarpal past a location of the fracture guided by the guidewire;
rotatably driving the cannulated intramedullary implant into the intramedullary canal guided by the guidewire, the implant comprising a headless shaft having a trailing end and a leading end, the shaft comprising a thread of a constant pitch, wherein a first portion of the shaft closer to the trailing end has a greater outer diameter and a second portion of the shaft closer to the leading end has a smaller outer diameter, and wherein threads on the first portion engage bone material in a larger distal portion of the metacarpal and threads on the second portion engage bone material in a narrower proximal or mid portion of the metacarpal such that the cannulated intramedullary implant is anchored in the metacarpal; and
removing the guidewire.

12. The method of claim 11, wherein the cannulated intramedullary implant is inserted with the leading end entering the opening on the intramedullary canal first.

13. The method of claim 11, wherein the threads on the first portion engages the metacarpal head.

14. The method of claim 11, wherein the shaft has a wall thickness so that the implant is strong enough to be rotatably driven into the metacarpal by a torque without being deformed.

15. The method of claim 11, wherein the threads on the first portion and the threads on the second portion are of the same thread height.

16. The method of claim 11, wherein rotatably driving the cannulated intramedullary implant is performed using a driver, the implant comprising a driving head at the trailing end, wherein the driving head is configured to receive the driver.

17. The method of claim 16, wherein the driver is a flat screwdriver, a Phillips screwdriver, a hex head, or an Allen wrench.

18. The method of claim 11, wherein the shaft further comprises a transition region, a root diameter of the transition region transitions from the greater outer diameter to the smaller outer diameter.

19. The method of claim 1, wherein the shaft comprises at least one cutting structure extending through part of the first portion and at least another cutting structure extending through part of the second portion.
